(19) United States
(12) Patent Application Publication
(10) Pub. No.: US 2007/0111932 A1
(43) Pub. Date: May 17, 2007

(54) Method of enhancing and/or inducing neuronal migration using erythropoietin

(75) Inventor: Linda B Andersen, Calgary (CA)

Correspondence Address: FISH & RICHARDSON P.C., PO BOX 1022, MINNEAPOLIS, MN, 55440-1022, US

(73) Assignee: Stem Cell Therapeutics Inc., Calgary (CA)

(21) Appl. No.: 10/558,661

(22) PCT Filed: Jul. 31, 2003
(86) PCT No.: PCT/CA03/01181

§ 371 Date: Jan. 25, 2007

(51) Int. Cl. A61K 38/18

(52) U.S. Cl. 514/12

(57) ABSTRACT

Methods are described for the enhancement and/or induction of migration of neural stem cells or neuronal progenitor cells. Multipotent neural stem cells are exposed to erythropoietin which enhances the migration of multipotent neural stem cells and neuronal progenitor cells. The erythropoietin may be exogenously applied to the multipotent neural stem cells, or alternatively, the cells can be subjected to hypoxic insult which induces the cells to express erythropoietin. In a preferred embodiment, additional growth factors, such as EGF and prolactin, are utilized.

### RELATED APPLICATIONS

This application claims the benefit of U.S. Provisional Application Ser. No. 60/399,395, filed Jul. 31, 2002. The entire disclosure of this priority application is hereby incorporated by reference.

### FIELD OF THE INVENTION

This invention relates to methods of enhancing and/or inducing the migration of multipotent neural stem cells and their progeny by exposing the stem cells and their progeny to erythropoietin. In a preferred embodiment, additional growth factors are also utilzied.

### REFERENCES

U.S. Pat. No. 4,703,008.

U.S. Pat. No. 5,128,242.

U.S. Pat. No. 5,198,542.

U.S. Pat. No. 5,208,320.

U.S. Pat. No. 5,326,860.

U.S. Pat. No. 5,441,868.

U.S. Pat. No. 5,547,935.

U.S. Pat. No. 5,547,993.

U.S. Pat. No. 5,621,080.

U.S. Pat. No. 5,623,050.

U.S. Pat. No. 5,750,376.

U.S. Pat. No. 5,801,147.

U.S. Pat. No. 5,955,346.

U.S. Pat. No. 6,165,783.

U.S. Pat. No. 6,191,106.

U.S. Pat. No. 6,242,563.

U.S. Pat. No. 6,294,346.

U.S. Pat. No. 6,376,218.

U.S. Pat. No. 6,429,186.

U.S. Pat. No. 6,618,698.

International PCT Application No. WO 93/01275.

International PCr Application No. WO 94/10292.

International PCT Application No. WO 03/040310.

S. A. Bayer, “Neuron production in the hippocampus and olfactory bulb of the adult rat brain: addition or replacement?” N.Y. Acad. Sci. 457:163-173 (1985).

S. Bernichtein et al., “S179D-human PRL, a pseudophosphorylated human PRL analog, is an agonist and not an antagonist, ” Endocrinology 142(9):3950-3963 (2001).

C. G. Craig et al., “In vivo growth factor expansion of endogenous subependymal neural precursor cell populations in adult mouse brain,” J. Neurosci. 16(8):2649-58 (1996).

C. R. Freed et al., “Survival of implanted fetal dopamine cells and neurologic improvement 12 to 46 months after transplantation for Parkinson's Disease,” N. Engl. J. Med. 327:1549-1555 (1992).

M. S. Kaplan, “Neurogenesis in the 3-month old rat visual cortex,” J. Comp. Neurol. 195:323-338 (1981)

D. van der Kooy and S. Weiss, “Why stem cells?” Science 287:1439-41 (2000).

M. J. Perlow et al., “Brain grafts reduce motor abnormalities produced by destruction of nigrostriatal dopamine system,” Science 204:643-647 (1979).

C. S. Potten and Loeffler, “Stem cells: attributes, cycles, spirals, pitfalls and uncertainties. Lessons for and from the Crypt,” Development 110:1001-1020 (1990).

P. Rakic, “Limits of neurogenesis in primates,” Science 227:1054-1056 (1985).

B. A. Reynolds and S. Weiss, “Generation of neurons and astrocytes from isolated cells of the adult mammalian central nervous system,” Science 255:1707-1710 (1992).

R. Rietze et al., “Mitotically active cells that generate neurons and astrocytes are present in multiple regions of the adult mouse hippocampus,” J. Comp. Neurol. 424(3):397-408 (2000)

T. Shingo et al., “Erythropoietin regulates the in vitro and in vivo production of neuronal progenitors by mammalian forebrain neural stem cells,” J. Neurosci. 21(24):9733-9743 (2001).

D. D. Spencer et al. “Unilateral transplantation of human fetal mesencephalic tissue into the caudate nucleus of patients with Parkinson's Disease,” N. Engl. J. Med. 327:1541-1548 (1992).

H. Widner et al., “Bilateral fetal mesencephalic grafting into two patients with Parkinsonism induced by 1-methyl-phenyl-1,2,3,6-tetrahydropyridine (MPTP),” N. Engl. J. Med. 327:1556-1563 (1992).

All of the publications, patents, and patent applications cited in this application are hereby incorporated by reference in their entirety to the same extent as if the disclosure of each individual publication, patent, or patent application was specifically and individually indicated to be incorporated by reference in its entirety.

### BACKGROUND OF THE INVENTION

Neurogenesis in mammals is largely complete early in the postnatal period. While it was previously thought that cells of the adult mammalian central nervous system (CNS) have little or no ability to undergo mitosis and generate new neurons, recent studies have demonstrated that the mature nervous system does have some limited capability to produce new neurons. (Craig et al., 1996; Rietze et al., 2000; review in van der Kooy and Weiss, 2000). Several mammalian species (e.g., rats) exhibit the limited ability to generate new neurons in restricted adult brain regions such as the dentate gyrus and olfactory bulb (Kaplan, 1981; Bayer, 1985). However, the generation of new CNS neurons in adult primates does not normally occur (Rakic, 1985). This relative inability to produce new neural cells in most mammals (and especially primates) may be advantageous for long-term memory retention; however, it is a distinct disadvantage when the need to replace lost neuronal cells arises due to an injury or disease.

The role of neural stem cells in the adult is to replace cells that are lost by natural cell death, injury or disease. Until recently, the low turnover of cells in the mammalian CNS together with the inability of the adult mammalian CNS to generate new neuronal cells in response to the loss of cells following an injury or disease had led to the assumption that the adult mammalian CNS does not contain multipotent neural stem cells. The critical identifying feature of a stem cell is its ability to exhibit self-renewal or to generate more of itself. The simplest definition of a stem cell would be a cell with the capacity for self-maintenance. A more stringent (but still simplistic) definition of a stem cell is provided by Potten and Loeffler (1990) who have defined stem cells as “undifferentiated cells capable of a) proliferation, b) self-maintenance, c) the production of a large number of differentiated functional progeny, d) regenerating the tissue after injury, and e) a flexibility in the use of these options.”

CNS disorders encompass numerous afflictions such as neurodegenerative diseases (e.g., Alzheimer's and Parkinson's), brain injury (e.g., stroke, head injury, cerebral palsy) and a large number of CNS dysfunctions (e.g., depression, epilepsy, and schizophrenia). In recent years, neurodegenerative disease has become an important concern due to the expanding elderly population which is at the greatest risk for these disorders. These diseases, which include Alzheimer's Disease, Parkinson's Disease, Huntington's Disease, Multiple Sclerosis (MS), and Amyotrophic Lateral Sclerosis, have been linked to the degeneration of neuronal cells in particular locations of the CNS, leading to the inability of these cells or the brain region to carry out their intended function.

Degeneration in a brain region known as the basal ganglia can lead to diseases with various cognitive and motor symptoms, depending on the exact location. The basal ganglia consists of many separate regions, including the striatum (which consists of the caudate and putamen), the globus pallidus, the substantia nigra, substantia innominate, ventral pallidum, nucleus basalis of Meynert, ventral tegmental area and the subthalamic nucleus. Many motor deficits are a result of neuronal degeneration in the basal ganglia. Huntington's Chorea is associated with the degeneration of neurons in the striatum, which leads to involuntary jerking movements in the host. Degeneration of a small region called the subthalamic nucleus is associated with violent flinging movements of the extremities in a condition called ballismus, while degeneration in the putamen and globus pallidus is associated with a condition of slow writhing movements or athetosis. In the case of Parkinson's Disease, degeneration is seen in another area of the basal ganglia, the substantia nigra pars compacta. This area normally sends dopaminergic connections to the dorsal striatum which are important in regulating movement. In the case of Alzheimer's Disease, there is a profound cellular degeneration of the forebrain and cerebral cortex. In addition, upon closer inspection, a localized degeneration in an area of the basal ganglia, the nucleus basalis of Meynert, appears to be selectively degenerated. This nucleus normally sends cholinergic projections to the cerebral cortex which are thought to participate in cognitive functions including memory.

Other forms of neurological impairment can occur as a result of neural degeneration, such as cerebral palsy, or as a result of CNS trauma, such as stroke and epilepsy.

In addition to neurodegenerative diseases, brain injuries often result in the loss of neurons, the inappropriate functioning of the affected brain region, and subsequent behavior abnormalities. Probably the largest area of CNS dysfunction (with respect to the number of affected people) is not characterized by a loss of neural cells but rather by an abnormal functioning of existing neural cells. This may be due to inappropriate firing of neurons, or the abnormal synthesis, release, and/or processing of neurotransmitters. These dysfunctions may be the result of well studied and characterized disorders such as depression and epilepsy, or less understood disorders such as neurosis and psychosis.

Other forms of neurological impairment can occur as a result of neural degeneration, such as amyotrophic lateral sclerosis and cerebral palsy, or as a result of CNS trauma such as stroke and epilepsy.

Demyelination of central and peripheral neurons occurs in a number of pathologies and leads to improper signal conduction within the nervous system. Myelin is a cellular sheath, formed by glial cells, that surrounds axons and axonal processes that enhances various electrochemical properties and provides trophic support to the neuron. Myelin is formed by Schwann cells in the peripheral nervous system and by oligodendrocytes in the central nervous system. Among the various demyelinating diseases, MS is the most notable.

To date, treatment for CNS disorders has been primarily via the administration of pharmaceutical compounds. Unfortunately, this type of treatment has been fraught with many complications including limited ability to transport drugs across the blood-brain barrier and drug-tolerance acquired by patients to whom these drugs are administered long-term. For instance, partial restoration of dopaminergic activity in Parkinson's patients has been achieved with levodopa, which is a dopamine precursor able to cross the blood-brain barrier. However, patients become tolerant to the effects of levodopa, and therefore, steadily increasing dosages are needed to maintain its effects. In addition, there are a number of side effects associated with levodopa such as increased and uncontrollable movement.

Recently, the concept of neurological tissue grafting has been applied to the treatment of neurological diseases such as Parkinson's Disease. Neural grafts may avert the need not only for constant drug administration, but also for complicated drug delivery systems which arise due to the blood-brain barrier. However, there are limitations to this technique as well. First, cells used for transplantation which carry cell surface molecules of a differentiated cell from another host can induce an immune reaction in the host. In addition, the cells must be at a stage of development where they are able to form normal neural connections with neighboring cells. For these reasons, initial studies on neurotransplantation centered on the use of fetal cells. Several studies have shown improvements in patients with Parkinson's Disease after receiving implants of fetal CNS tissue. Implantation of embryonic mesencephalic tissue containing dopamine cells into the caudate and putamen of human patients was shown by Freed et al. (1992) to offer long-term clinical benefit to some patients with advanced Parkinson's Disease. Similar success was shown by Spencer et al. (1992). Widner et al. (1992) have shown long-term functional improvements in patients with N-methyl-4-phenyl-1,2,3,6-tetrahydropyridine (MPTP)-induced Parkinsonism that received bilateral implantation of fetal mesencephalic tissue. Perlow et al. (1979) describe the transplantation of fetal dopaminergic neurons into adult rats with chemically induced nigrostriatal lesions. These grafts showed good survival, axonal outgrowth and significantly reduced the motor abnormalities in the host animals. A further discussion of tissue transplantation techniques and drawbacks can be found in U.S. Pat. No. 6,294,346 B1.

While the studies noted above are encouraging, the use of large quantities of aborted fetal tissue for the treatment of disease raises ethical considerations and political obstacles. There are other considerations as well. Fetal CNS tissue is composed of more than one cell type, and thus is not a well-defined source of tissue. In addition, there are serious doubts as to whether an adequate and constant supply of fetal tissue would be available for transplantation. For example, in the treatment of MPTP-induced Parkinsonism (Widner, 1992) tissue from 6 to 8 fresh fetuses were required for implantation into the brain of a single patient. There is also the added problem of the potential for contamination during fetal tissue preparation. Moreover, the tissue may already be infected with a bacteria or virus, thus requiring expensive diagnostic testing for each fetus used. However, even diagnostic testing might not uncover all infected tissue. For example, the successful diagnosis of HIV-free tissue is not guaranteed because antibodies to the virus are generally not present until several weeks after infection.

While currently available transplantation approaches represent a significant improvement over other available treatments for neurological disorders, they suffer from significant drawbacks. The inability in the prior art of the transplant to fully integrate into the host tissue, and the lack of availability of neuronal cells in unlimited amounts from a reliable source for grafting are, perhaps, the greatest limitations of neurotransplantation. A well-defined, reproducible source of neural cells is currently available. It has been discovered that multipotent neural stem cells, capable of producing progeny that differentiate into neurons and glia, exist in adult mammalian neural tissue. (Reynolds and Weiss, 1992). Methods have been provided for the proliferation of these stem cells to provide large numbers of neural cells that can differentiate into neurons and glia (See U.S. Pat. No. 5,750,376, and International Application No. WO 93/01275). Various factors can be added to neural cell cultures to influence the make-up of the differentiated progeny of multipotent neural stem cell progeny, as disclosed in published PCT application WO 94/10292. Additional methods for directing the differentiation of stem cell progeny were disclosed in U.S. Pat. No. 6,165,783 utilizing erythropoietin and various growth factors.

Thus, the repair of damaged neural tissue may potentially be replaced in a relatively non-invasive fashion, by inducing neural cells to proliferate and differentiate into neurons, astrocytes, and oligodendrocytes in vivo, averting the need for transplantation. However, simply inducing neural cells to proliferate and differentiate is not always sufficient to treat a neurodegenerative disease or brain injury if the new neurons are not able to reach the lesioned or damaged area. During development, neurons in many regions of the brain are directed to their appropriate destinations by migrating along radial glia. For example, developing neurons migrate outward from the ventricular zone to the cortical plate. As many neural stem cells in the adult nervous system are in the localized areas, which may be remote from the affected areas, it is particularly desirable to be able to elicit migration of these cells to other affected areas of the brain to replace lost neurons, e.g., the basal ganglia in Parkinson's Disease.

### SUMMARY OF THE INVENTION

Accordingly, a major object of the present invention is to provide both in vivo and in vitro techniques of enhancing or inducing migration of multipotent neural stem cells or multipotent neural stem cell progeny.

The current invention provides a method of enhancing or inducing the migration of multipotent neural stem cell and/or multipotent neural stem cell progeny in a subject comprising administering erythropoietin to a subject in an amount effective to enhance neural stem cell migration. In a preferred embodiment, at least one other growth factor besides erythropoietin is administered. In a particularly preferred embodiment, the other growth factor is epidermal growth factor. In another embodiment, the other growth factor is prolactin.

The erythropoietin and growth factors can be administered in a different order. In one embodiment, the erythropoietin is administered concurrently with at least one other growth factor. In an alternative embodiment, the erythropoietin is administered sequentially with at least one other growth factor. In a preferred embodiment, at least one other growth factor is administered prior to the administration of erythropoietin. In an alternative embodiment, the at least one other growth factor is administered after the erythropoietin.

In one embodiment, the subject is suffering from a neurodegenerative disease or brain injury. In various embodiments, the subject is suffering from Alzheimer's Disease, Multiple Sclerosis, Huntington's Disease, Amyotrophic Lateral Sclerosis, Parkinson's Disease, surgery, stroke, a physical accident, depression, epilepsy, neurosis, or psychosis. In a particularly preferred embodiment, the subject is suffering from a stroke.

In an embodiment of the invention, the multipotent neural stem cells and/or progeny migrate towards a lesioned or damaged area of the brain of the subject. In a particularly preferred embodiment, the multipotent neural stem cells and/or progeny migrate to the basal ganglia. In one embodiment, the subject is a mammal. In a preferred embodiment, the subject is a human. In a particularly preferred embodiment, the mammal is an adult. In another embodiment, the multipotent neural stem cells and/or progenitor cells which are derived from the multipotent neural stem cells are transplanted into the subject. In a preferred embodiment, the multipotent neural stem cells and/or progenitor cells are incubated with erythropoietin and at least one growth factor before being transplanted into the subject.

Another aspect of the invention provides a method of enhancing or inducing the migration of multipotent neural stem cells and/or multipotent neural stem cell progeny comprising exogenously adding to the multipotent neural stem cells and/or multipotent neural stem cell progeny an amount of erythropoietin effective to cause the multipotent neural stem cells and/or multipotent neural stem cell progeny to migrate. In a preferred embodiment, at least one other growth factor is added. In a particularly preferred embodiment, the other growth factor is epidermal growth factor. In another embodiment, the at least one other growth factor is prolactin.

In another embodiment, the erythropoietin is added concurrently with the at least one other growth factor. In an alternative embodiment, the erythropoietin is added sequentially with the at least one other growth factor. In a particularly preferred embodiment, the other growth factor is added prior to the addition of erythropoietin. In another embodiment, the other growth factor is added after the addition of erythropoietin.

Another aspect of the invention provides a method for enhancing or inducing migration of multipotent neural stem cells and/or multipotent neural stem cell progeny, comprising exposing said multipotent neural stem cells and/or multipotent stem cell progeny to hypoxic conditions to induce expression of erythropoietin in order to enhance or induce migration. In a preferred embodiment, at least one other growth factor is exogenously added. In a particularly preferred embodiment, the other growth factor is epidermal growth factor. In another embodiment, the other growth factor is prolactin. In one embodiment, the other growth factor is added to said multipotent neural stem cells and/or multipotent neural stem cell progeny concurrently with hypoxic conditions. In an alternative embodiment, the other growth factor is added to the multipotent neural stem cells and/or multipotent neural stem cell progeny sequentially with hypoxic conditions. In a particularly preferred embodiment, the other growth factor is added to the multipotent neural stem cells and/or multipotent neural stem cell progeny prior to exposure to hypoxic conditions. In another embodiment, the other growth factor is added after exposure to hypoxic conditions.

### BRIEF DESCRIPTION OF THE DRAWINGS

FIG. 1. Distribution of total BrdU+ cells between the subventricular zone (SVZ) and the striatum (Str) in mice lesioned with ibotenic acid and treated with epidermal growth factor (EGF) and Erythropoietin (Epo). When administered to animals treated with EGF, Epo enhanced the number of neural progenitors in the stratium. (*p<0.05).

FIG. 2. Number of NeuN+/BrdU+ cells (mature neurons) in the striatum of mice lesioned with ibotenic acid and treated with EGF and Epo. EGF enhanced the number of NeuN+/BrdU+ cells in the striatum. When administered to animals treated with EGF, Epo further enhanced this effect.

FIG. 3. 3A: Number of Dcx+/BrdU+ cells (immature neurons or neuronal precursors) in the subventricular zone (SVZ) in mice lesioned with ibotenic acid and treated with EGF and Epo. 3B: Number of Dcx+/BrdU+ cells (immature neurons or neuronal precursors) in the striatum of mice lesioned with ibotenic acid and treated with EGF and Epo. When administered to animals treated with EGF, Epo enhanced migration of neuronal precursors into the damaged striatum. (*p<0.05).

### DETAILED DESCRIPTION OF THE INVENTION

The present invention relates to a method of enhancing or inducing migration of multipotent neural stem cells or multipotent neural stem cell progeny by utilizing erythropoietin in conjunction with at least one other growth factor.

Prior to describing the invention in further detail, the terms used in this application are defined as follows unless otherwise indicated.

As used herein, the term “multipotent neural stem cell” or “neural stem cell” refers to an undifferentiated cell which is capable of self-maintenance. Thus, in essence, a stem cell is capable of dividing without limit. “Progenitor cells” are non-stem cell progeny of a multipotent neural stem cell. A distinguishing feature of a progenitor cell is that, unlike a stem cell, it has limited proliferative ability and thus does not exhibit self-maintenance. It is committed to a particular path of differentiation and will, under appropriate conditions, eventually differentiate. A neuronal progenitor cell is capable of a limited number of cell divisions before giving rise to differentiated neurons. A glial progenitor cell likewise is capable of a limited number of cell divisions before giving rise to astrocytes or oligodendrocytes. A neural stem cell is multipotent because its progeny include both neuronal and glial progenitor cells and thus is capable of giving rise to neurons, astrocytes, and oligodendrocytes. Multipotent neural stem cell progeny include neuronal precursor cells, glial precursor cells, neurons, and glial cells.

A “neurosphere” is a group of cells derived from a single neural stem cell as the result of clonal expansion. Primary neurospheres may be generated by plating as primary cultures brain tissue which contains neural stem cells. The method for culturing neural stem cells to form neurospheres has been described in, e.g., U.S. Pat. No. 5,750,376. Secondary neurospheres may be generated by dissociating primary neurospheres and allowing the individual dissociated cells to form neurospheres again.

By “growth factor” is meant a substance that affects the growth of a cell or an organism, including proliferation, differentiation, and increases in cell size. A growth factor is a polypeptide which shares substantial sequence identity with a native mammalian growth factor and possesses a biological activity of the native mammalian growth factor. In a preferred embodiment, the native mammalian growth factor is a native human growth factor. Having a biological activity of a native mammalian growth factor means having at least one activity of a native mammalian growth factor, such as binding to the same receptor as a particular native mammalian growth factor binds and/or eliciting proliferation and/or differentiation and/or changes in cell size. Preferably, the growth factor binds to the same receptor as a particular native mammalian growth factor. This includes functional variants of the native mammalian growth factor.

A polypeptide which shares substantial sequence identity with a native mammalian growth factor is at least about 30% identical to the native mammalian growth factor at the amino acid level. The growth factor is preferably at least about 40%, more preferably at least about 60%, and most preferably about 60% identical to the native mammalian growth factor at the amino acid level. Thus, the term growth factor encompasses analogs which are deletional, insertional, or substitutional mutants of a native mammalian growth factor. Furthermore, the term growth factor encompasses the growth factors from other species and naturally occurring and synthetic variants thereof.

Erythropoietin (Epo) is a growth factor. Other exemplary growth factors that may be used in conjunction with Epo in embodiments of the present invention include, inter alia, platelet-derived growth factor (PDGF), epidermal growth factor (EGF), insulin-like growth factor-1 and -2 (IGF-1, IGF-2), transforming growth factors α and β (TGF-α, TGF-β), acidic and basic fibroblast growth factors (a-FGF/FGF-2, b-FGF/FGF-2), interleukins 1, 2, 6, and 8 (IL-1, IL-2, IL-6, IL-8), nerve growth factor (NGF), brain-derived neurotrophic factor (BDNF), neurotrophin-3 (NT-3), interleukin-3, hematopoietic colony stimulating factors (CSFs), amphiregulin, interferon-γ (INF-γ), thyrotropin releasing hormone (TRH), pituitary adenylate cyclase activating polypeptide (PACAP), and prolactin. In a preferred embodiment, Epo is used in conjunction with EGF. In another embodiment, Epo is used in conjunction with prolaction.

It should be noted that variants or analogs of these agents, which share a substantial identity with a native mammalian growth factor listed above and are capable of binging the receptor for a native mammalian growth factor, can be used in the present application. For example, there are two forms of mammalian PACAP, PACAP38 and PACAP27. Any variant or analog that is capable of binding to a receptor for a native mammalian PACAP and shares a substantial sequence identity with either PACAP38 or PACAP27 is suitable for use in the present invention. Particularly useful are the analogs and variants disclosed in, e.g., U.S. Pat. Nos. 5,128,242; 5,198,542; 5,208,320; 5,326,860; 5,801,147, and 6,242,563.

Similarly, EGF variants or analogs, which share a substantial identity with a native mammalian EGF and are capable of binding to a receptor for the native mammalian EGF, can be used in the present application. These EGF variants and analogs include, but are not limited to, the recombinant modified EGF having a deletion of the two C-terminal amino acids and a neutral amino acid substitution at position 51, such as asparagine, glutamine, serine, or alanine (particularly EGF51N or EGF51Q, having asparagine (N) or glutamine (Q) at position 51, respectively; WO 03/040310); the EGF mutein (EGF-X16) in which the His residue at position 16 is replaced with a neutral or acidic amino acid (U.S. Pat. No. 6,191,106); the 52-amino acid deletion mutant of EGF which lacks the amino terminal residue of the native EGF (EGF-D); the EGF deletion mutant in which the amino terminal residue as well as the two C-terminal residues (Arg-Leu) are deleted (EGF-B); the EGF-D in which the Met residue at position 21 is oxidized (EGF-C); the EGF-B in which the Met residue at position 21 is oxidized (EGF-A); heparin-binding EGF-like growth factor (HB-EGF); betacellulin; amphiregulin; neuregulin; or a fusion protein comprising any of the above. Other usefull EGF analogs or variants are described in WO 03/040310, and U.S. Pat. Nos. 6,191,106 and 5,547,935.

Specifically included as prolactins are the natarally occurring prolactin variants, prolactin-related protein, placental lactogens, S179D-human prolactin (Bernichtein et al., 2001), prolactins from various mammalian species, including, but not limited to, human, other primates, rat mouse, sheep, pig, and cattle, and the prolactin mutants described in U.S. Pat. Nos. 6,429,186 and 5,955,346.

“Erythropoietin” refers to a polypeptide that shares substantial sequence similarity with native mammalian erythropoietin and possesses a biological activity of the native mammalian erythropoietin, including recombinant erythropoietin or epoietin. Having a biological activity of native mammalian erythropoietin means having at least one activity of a native mammalian erythropoietin, such as binding to the same receptor as the native mammalian erythropoietin binds and/or eliciting proliferation and/or differentiation, and/or changes in cell size. Preferably, the polypeptide binds to a native mammalian Epo receptor. This includes functional variants of the native mammalian erythropoietin. The native human eryihropoietin is a glycoprotein of 165 or 166 amino acids (C-terninal arginine is removed in post-translational modification) and an approximate molecular weight of 30-40 kDa.

Erythropoietin can be generated or synthesized using genetic engineering techniques such as those found in U.S. Pat. Nos. 4,703,008; 5,441,868; 5,547,993, 5,621,080, 6,618,698, and 6,376,218. A polypeptide which shares “substantial sequence similarity” with the native mammalian erythropoietin is at least about 30% identical with native mammalian erythropoietin at the amino acid level. The erythropoietin is preferably about 40%, more preferably about 60%, yet more preferably at least about 70%, and most preferably, at least about 80% identical with the native mammalian erythropoietin at the amino acid level. Thus, the term erythropoietin encompasses erydiropoietin analogs which are deletional, insertional, or substitutional mutants of the native mammalian erytiropoietin. Furthermore, the term erythropoietin encompasses erythropoietins from other species and the naturally occurring and synthetic variants thereof.

“Percent identity” or “% identity” refers to the percentage of amino acid sequence in a protein or polypeptide which are also found in a second sequence when the two sequences are aligned. Percent identity can be determined by any methods or algorithms established in the art, such as LALIGN or BLAST.

A polypeptide possesses the “biological activity” of a growth factor, including erydiropoeitin, if it is capable of exerting any of the biological activities of the native mammalian growth factor or being recognized by a polyclonal or monoclonal antibody raised against the native mammalian growth factor. Preferably, the polypeptide is capable of specifically binding to the receptor for the native growth factor in a receptor binding assay.

“Hypoxic conditions” or “hypoxia” refers to a decrease in normal or optimal oxygen conditions for a cell or an organism. Normal or optimal oxygen concentration is 135 mm Hg or 95% air/5% CO₂. Standard hypoxic conditions comprise an oxygen concentration of about 30-40 mm Hg.

“Migration” refers to the movement of a cell from one location to another. Thus, a substance that “enhances” migration increases the speed, distance, or number of cells moving from one location to another over the speed, distance, or number of cells moving in the absence of the substance. For instance, the Example below demonstrated that the distance traveled by multipotent neural stem cells and/or multipotent neural stem cell progeny is much greater with Epo and EGF compared to either of these alone. A substance that “induces” migration elicits migration when it would not otherwise occur in the absence of the substance. The present invention can be used to enhance or induce migration of neurons to damaged areas of the CNS.

A “neurodegenerative disease or condition” is a disease or a medical condition associated with neuron loss or dysfunction. Examples of neurodegenerative diseases or conditions include neurodegenerative diseases, brain injuries or CNS dysfunctions. Neurodegenerative diseases include, e.g., Alzheimer's Disease, Multiple Sclerosis, Huntington's Disease, Amyotrophic Lateral Sclerosis, and Parkinson's Disease. Brain injuries include, e.g., injuries to the nervous system due to surgery, stroke, and physical accidents. CNS dysfunctions include, e.g., depression, epilepsy, neurosis, and psychosis.

“Treating or ameliorating” means the reduction or complete removal of the symptoms of a disease or medical condition.

An “effective amount” is an amount of a therapeutic agent sufficient to achieve the intended purpose. For example, an effective amount of a growth factor or erythropoietin to enhance the migration of neural stem cells is an amount sufficient, in vivo or in vitro, to result in an enhancement in migration of neural stem cells over the speed, distance, or number in the absence of the growth factor or erythropoietin. An effective amount of a growth factor or erythropoietin to treat or ameliorate a neurodegenerative disease or condition is an amount of the growth factor or erythropoietin sufficient to reduce or remove the symptoms of the neurodegenerative disease or condition. The effective amount of a given therapeutic agent will vary with factors such as the nature of the agent, the route of administration, the size and species of the animal or subject to receive the therapeutic agent, and the purpose of administration. The effective amount in each individual case may be determined empirically by a skilled artisan according to established methods in the art.

### Detailed Description

In the present invention, a method of enhancing or inducing the migration of neural stem cells and/or their progeny was discovered. As discussed in more detail in the Example below, Epo was able to enhance the speed, number, and distance of migration of neural stem cells and/or their progeny. Preferably, at least one other growth factor is also used. For example, when animals with striatal lesions were treated with Epo and EGF, greater numbers of newly generated cells were discovered in the striatum. Additionally, more of the newly generated cells adopted a neuronal phenotype in the damaged striatum.

Various embodiments of the present invention are possible. In addition to EGF, other growth factors such as those described above can be used with Epo to enhance or induce migration of neural stem cells and/or their progeny. For instance, prolactin is another preferred embodiment of the present invention. When other growth factors are administered or added in conjunction with Epo, the order of administration or addition can be varied. Epo and the other growth factor can be administered or added sequentially or simultaneously. When added sequentially, Epo can be administered or added before or after the other growth factor.

The multipotent stem cells and/or their progeny can be induced to migrate or the migration to various areas of the brain can be enhanced. Although the Example below shows the migration of cells from the SVZ to the striatum, other embodiments are also contemplated. For example, the migration of multipotent neural stem cells or their progeny can be enhanced towards other areas of the basal ganglia or any other damaged area of the brain.

The present method can be practiced in vivo or in vitro. For in vivo administration, compositions containing Epo and/or other growth factors can be delivered via any route known in the art, such as orally, or parenterally, e.g., intravascularly, intramuscularly, transdermally, subcutaneously, or intraperitoneally. In a preferred embodiment, the composition is administered parenterally. Alternatively, the composition is delivered directly to the CNS. Direct administration into the CNS can be accomplished via delivery into a ventricle, such as the lateral ventricle.

According to embodiments of the invention, Epo and other growth factors may be administered in vivo to treat subjects suffering from neurodegenerative diseases, brain injuries, or CNS dysfunctions. Alzheimer's Disease, Huntington's Disease, and Parkinson's Disease, inter alia, may be treated according to various embodiments of the invention. Alternatively, the subject may be suffering from a stroke. Because of the prevalence of neurodegenerative disease in adults, the preferred subject is an adult human. However, it is contemplated that younger subjects may also suffer from neurodegenerative disease, or more commonly, traumatic brain injury, and thus will benefit from the present invention. Additionally, while humans are particularly preferred subjects, other species, such as those kept as pets, may also be treated according to an embodiment of the invention. Subjects may be treated with Epo and/or other growth factors, or neural stem cells may be exogenously treated and then transplanted into the subject. A combination of these approaches is also possible.

The present invention can be used in vitro. Multipotent neural stem cells can be obtained from embryonic, juvenile, or adult mammalian neural tissue (e.g., mouse and other rodents, and humans and other primates) or from other sources as described in U.S. Pat. No. 6,294,346 B1. Multipotent neural stem cells can be induced to proliferate in vitro or in vivo using the methods disclosed in published PCT application WO 93/01275 and U.S. Pat. Nos. 5,750,376 and 6,294,346 B1. Briefly, the administration of one or more growth factors can be used to induce the proliferation and differentiation of multipotent neural stem cells. Preferred proliferation-inducing growth factors include epidermal growth factor (EGF), amphiregulin, acidic fibroblast growth factor (AFGF or FGF-1), basic fibroblast growth factor (bFGP or FGF-2), transforming growth factor alpha (TGF-α), and combinations thereof. For the proliferation of multipotent neural stem cells in vitro, neural tissue is dissociated and the primary cell cultures are cultured in a suitable culture medium, such as the serum-free defined medium described U.S. Pat. No. 6,165,783. A suitable proliferation-inducing growth factor, such as EGF (20 ng/ml) is added to the culture medium to induce multipotent neural stem cell proliferation. In addition to proliferation-inducing growth factors, other growth factors may be added to the culture medium that influence proliferation and differentiation of the cells, including nerve growth factor (NGF), platelet-derived growth factor (PDGF), thyrotropin releasing hormone (TRF), transforming growth factor betas (TGF-βs), insulin-like growth factor (IGF-1) and the like.

In the absence of substrates that promote cell adhesion (e.g. ionically charged surfaces such as poly-L-lysine and poly-L-ornithine coated and the like), multipotent neural stem cell proliferation can be detected by the formation of clusters of undifferentiated neural cells termed “neurospheres,” which after several days in culture, lift off the floor of the culture dish and float in suspension. Each neurosphere results from the proliferation of a single multipotent neural stem cell and is comprised of daughter multipotent neural stem cells and neural progenitor cells. The neurospheres can be dissociated to form a suspension of undifferentiated neural cells and transferred to fresh growth-factor containing medium. This re-initiates proliferation of the stem cells and the formation of new neurospheres. In this manner, an unlimited number of undifferentiated neural stem cell progeny can be produced by the continuous culturing and passaging of the cells in suitable culture conditions.

Various procedures are disclosed in WO 94/10292 and U.S. Pat. Nos. 5,750,376 and 6,294,346 B1 which can be used to induce the proliferated neural stem cell progeny to differentiate into neurons, astrocytes and oligodendrocytes. Various methods of assessing differentiation of a particular cell type, e.g., using immunochemistry, are described in U.S. Pat. No. 6,294,346 B1.

The ability to manipulate the fate of the differentiative pathway of the multipotent neural stem cell progeny to produce more neuronal progenitor cells and neurons is beneficial. Cell cultures with an enriched neuronal-progenitor cell and/or neuron population can be used for transplantation to treat various neurological injuries, diseases or disorders. The neuronal progenitor cells or neurons or a combination thereof can be harvested and transplanted into a patient needing neuronal augmentation. Neuronal progenitor cells are particularly suitable for transplantation because they are still undifferentiated and, unlike differentiated neurons, there are no branched processes which can be damaged during transplantation procedures. Once transplanted, the neuronal progenitor cells can migrate to a damaged area of the brain and differentiate in situ into new, functioning neurons. Suitable transplantation methods are known in the art and are disclosed in U.S. Pat. Nos. 5,750,376 and 6,294,346 B1.

Alternatively, a patient's endogenous multipotent neural stem cells could be induced to proliferate, migrate, and differentiate in situ by administering to the patient a composition comprising one or more growth factors, which induces the patient's neural stem cells to proliferate, and Epo, which instructs the proliferating neural stem cells to produce neuronal progenitor cells which eventually differentiate into neurons and enhances and/or induces migration to other brain regions. Suitable methods for administering a composition to a patient which induces the in situ proliferation of the patient's stem cells are disclosed in U.S. Pat. Nos. 5,750,376 and 6,294,346 B1.

### EXAMPLES

An in vivo mouse model of neurodegenerative disease and the use of Epo and EGF to induce neuronal migration.

EGF has been shown to induce proliferation of neural stem cells in the subventricular zone (SVZ). Previously, it was demonstrated that after a unilateral striatal lesion, newly-generated cells from both hemispheres migrated towards the damaged area in response to EGF. Epo is able to direct neural stem cells to differentiate into neuronal precursors. (Shingo et al., 2001). A mouse model of neurodegenerative disease was used to determine the effects of EGF and Epo on neural stem cell migration. Following an injury to elicit neurodegeneration, mice were infused with epidermal growth factor (EGF) and erythropoietin to induce proliferation, differentiation, and migration of endogenous neural precursor cells.

Adult male CD-1 mice were given an injection of ibotenic acid (4.0 μg in 1.6 μl total volume) into the medial striatum. Within one week, many of the striatal neurons within the lesion area had degenerated. At this stage, a miniosmotic pump filled with EGF (33 μ/ml) was inserted beneath the skin above the shoulders. A small hole was drilled through the skull and a cannula was secured to the skull with dental cement. The pump was connected via tubing to the cannula, which delivers EGF into the lateral ventricle of the brain for a period of seven (7) days.

At the end of the seven day period, mice were injected once every two (2) hours over a ten-hour period with bromodeoxyuridine (BrdU)(Sigma Chemical Co.), a marker for cell division. On the same day, a small incision was made directly above the pump on the back, the tubing was cut, and the EGF pump was replaced with another pump containing erythropoietin (1000 IU/ml). After seven days of Epo delivery, the cannula was removed from the skull and the wound was closed. The mice were sacrificed immediately following Epo delivery. A series of control mice were infused with the delivery vehicle only, mouse serum albumin.

The mice were sacrificed via transcardial perfusion under anesthesia whereby the brain is fixed with 4% paraformaldehyde. The brains were removed and subjected to a series of postfixation and cryoprotection steps before being frozen. The brains were cut into 12 μm sections and immunostained with markers for migrating immature neurons Doublecortin (Dcx) (Chemicon)) or mature neurons (NeuN (Chemicon)), and for proliferating cells (BrdU). Once brains were sectioned and stained, total BrdU and NeuN/BrdU and Dcx/BrdU cells were counted on every tenth section through the entire forebrain. The data presented below were the results of three independent experiments.

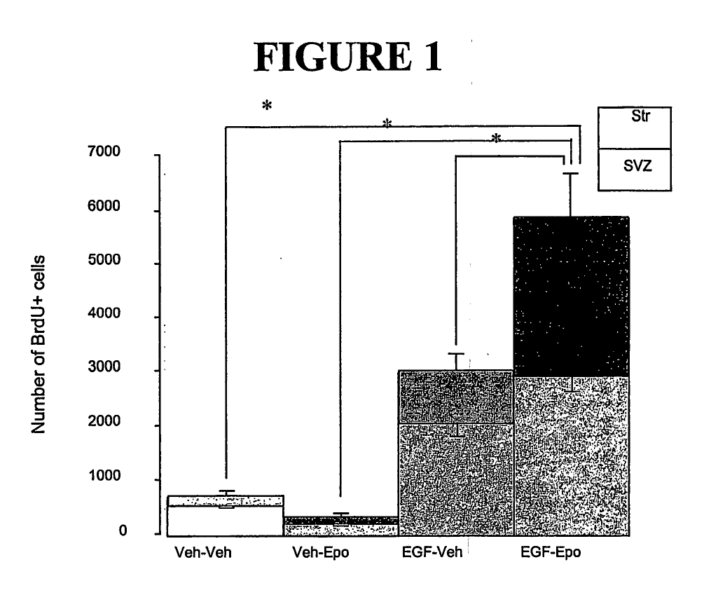

Infusion of EGF followed by Epo results in a greater number of newly generated cells (BrdU+) in the striatum compared to EGF alone. FIG. 1 shows the distribution of total BrdU+ cells between the subventricular zone (SVZ) and the striatum (Str). These data indicate that Epo enhances the numbers of neural progenitors in the striatum.

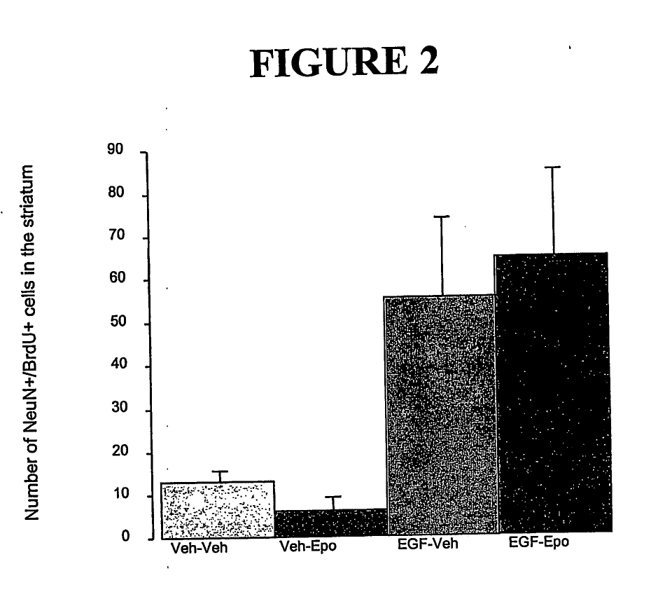

Newly generated cells in the striatum adopted a neuronal phenotype in the damaged striatum. Some of the newly generated cells differentiated into mature neurons (NeuN+/BrdU+) regardless of the infusion conditions. As can be seen in FIG. 2, all of the NeuN+/BrdU+ mature neurons are found in the striatum, indicating that they may have migrated from the SVZ and differentiated in the striatum.

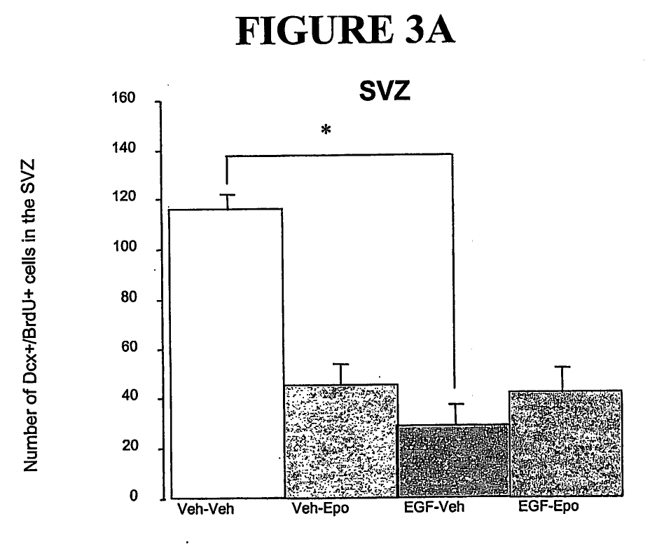

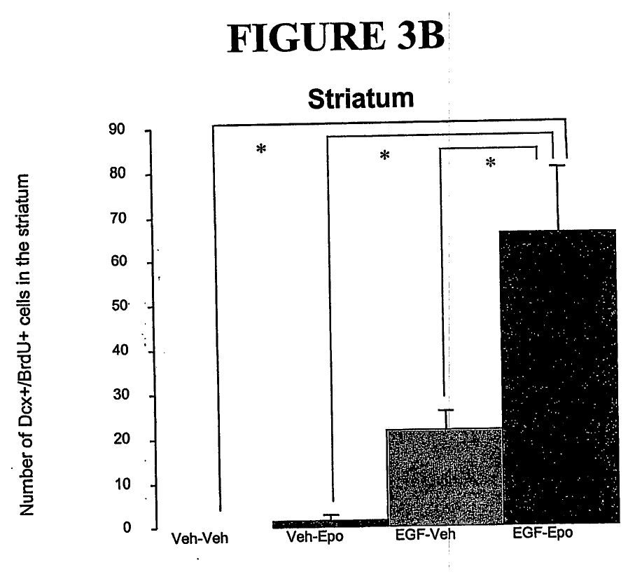

EGF followed by Epo infusion directs the migration of neuronal progenitors from the SVZ into the damaged striatum. In vehicle-only-infused mice, neuronal progenitors (Dcx+) remain in the SVZ after two weeks of treatment. The same results are seen in Vehicle-Epo-infused mice. In EGF-infused mice, neuronal progenitors moved laterally into the striatum. In EGF-Epo-infused mice most of the neuronal progenitors have migrated into the striatum. Newly generated BrdU+ cells outside the SVZ exhibited extending processes, indicating migration laterally into the striatum (data not shown). FIGS. 3A and 3B show the distribution of Dcx +/BrdU+ cells between the SVZ and the striatum, respectively. The distribution of cells between the SVZ and the striatum indicates, surprisingly, that Epo enhanced the migration of neuronal precursors into the damaged striatum.

Thus, Epo, when infused in combination with EGF, resulted in increased numbers of newly generated BrdU+ cells in the ibotenate-lesioned striatum, compared to those generated using EGF alone. These results show that Epo enhances both the number and migration rate of precursors from the lateral ventricle towards the lesioned striatum. The Epo-stimulated cells infiltrate the entire striatum indicating they have migrated from their origin in the SVZ. Epo promotes increased migration and survival/differentiation of newly generated neuronal precursors and thus will be useful in therapeutic strategies aimed at enhancing functional recovery from CNS injury or disease.

The above example is merely illustrative of the present invention and is considered to be in no way limiting. The skilled artisan will appreciate numerous variations of the present invention.

1. A method of enhancing multipotent neural stem cell and/or multipotent neural stem cell progeny migration in a subject comprising administering an erythropoietin and at least one other growth factor to a subject in an amount effective to enhance migration of multipotent neural stem cells and/or multipotent neural stem cell progeny.

2. The method of claim 1, wherein the at least one other growth factor is epidermal growth factor (EGF).

3. The method of claim 2, wherein the EGF is EGF51N or EGF51Q.

4. The method of claim 1, wherein the at least one other growth factor is prolactin.

5. The method of claim 1, wherein the erythropoietin is administered concurrently with the at least one other growth factor.

6. The method of claim 1, wherein the erythropoietin is administered sequentially with the at least one other growth factor.

7. The method of claim 1, wherein the at least one other growth factor is administered prior to the erythropoietin.

8. The method of claim 1, wherein the at least one other growth factor is administered after the erythropoietin.

9. The method of claim 1, wherein said subject is suffering from a neurodegenerative disease or brain injury.

10. The method of claim 9, wherein the subject is suffering from stroke.

11. The method of claim 1, wherein the multipotent neural stem cells and/or multipotent neural stem cell progeny migrate to the basal ganglia.

12. The method of claim 1, wherein the multipotent neural stem cells and/or multipotent neural stem cell progeny migrate towards a lesioned or damaged area of the brain of the subject.

13. The method of claim 1, wherein said subject is a human.

14. The method of claim 1, wherein the multipotent neural stem cells and/or progenitor cells which are derived from said multipotent neural stem cells are transplanted into said subject.

15. The method of claim 14, wherein said multipotent neural stem cells and/or progenitor cells are incubated with the erythropoietin and at least one other growth factor before being transplanted into said subject.

16. A method of inducing the migration of multipotent neural stem cells and/or multipotent stem cell progeny comprising exogenously adding to said multipotent neural stem cells and/or multipotent neural stem cell progeny an amount of an erythropoietin and at least one other growth factor effective to cause the migration of multipotent neural stem cells and/or multipotent neural stem cell progeny.

17. The method of claim 16, wherein the at least one other growth factor is epidermal growth factor (EGF).

18. The method of claim 17, wherein the EGF is EGF51N or EGF51Q.

19. The method of claim 16, wherein the at least one other growth factor is prolactin.

20. The method of claim 16, wherein the erythropoietin is added concurrently with the at least one other growth factor.

21. The method of claim 16, wherein the erythropoietin is added sequentially with the at least one other growth factor.

22. The method of claim 16, wherein the at least one other growth factor is added prior to the addition of the erythropoietin.

23. The method of claim 16, wherein the at least one other growth factor is added after the addition of the erythropoietin.

24. A method for inducing migration of multipotent neural stem cells and/or multipotent neural stem cell progeny, comprising exposing said multipotent neural stem cells and/or multipotent neural stem cell progeny to hypoxic conditions to induce expression of erythropoietin and exogenously adding at least one other growth factor in an amount effective to induce migration.

25. The method of claim 24, wherein said at least one other growth factor is epidermal growth factor (EGF).

26. The method of claim 25, wherein the EGF is EGF51N, or EGF51Q.

27. The method of claim 24, wherein the at least one other growth factor is prolactin.

28. The method of claim 24, wherein said at least one other growth factor is added to said multipotent neural stem cells and/or multipotent neural stem cell progeny concurrently with hypoxic conditions.

29. The method of claim 24, wherein said at least one other growth factor is added to said multipotent neural stem cells and/or multipotent neural stem cell progeny sequentially with hypoxic conditions.

30. The method of claim 24, wherein said at least one other growth factor is added to said multipotent neural stem cells and/or multipotent neural stem cell progeny prior to exposure to hypoxic conditions.

31. The method of claim 24, wherein said at least one other growth factor is added to said multipotent neural stem cells and/or multipotent neural stem cell progeny after exposure to hypoxic conditions.
